(19) United States
(12) Patent Application Publication
(10) Pub. No.: US 2013/0116469 A1
(43) Pub. Date: May 9, 2013

(54) PROCESS FOR PRODUCING N-ACYL AMINO ACIDS

(71) Applicant: Sumitomo Chemical Company, Limited, Tokyo (JP)

(72) Inventors: Kana MATSUMURA, Niihama-shi (JP); Toshiaki SUZUKI, Osaka-shi (JP)

(73) Assignee: SUMITOMO CHEMICAL COMPANY, LIMITED, Tokyo (JP)

(21) Appl. No.: 13/667,295

(22) Filed: Nov. 2, 2012

(30) Foreign Application Priority Data
Nov. 4, 2011 (JP) 2011-242148

(51) Int. Cl. C07C 51/12

(52) U.S. Cl. 562/518

(57) ABSTRACT

An object of the present invention is to provide a process for producing an N-acyl amino acid (1) in a good yield. The present invention provides a process for producing an N-acyl amino acid (1) by reacting an aldehyde compound (2), an amide compound (3), and carbon monoxide in the solvent in a reactor in the presence of a cobalt compound and hydrogen, characterized in the aldehyde compound (2), the amide compound (3) and the solvent are supplied to the reactor in which the solvent, the cobalt compound, hydrogen and carbon monoxide have been placed in advance.

This application claims priority to and the benefit of Japanese Patent Application No. 2011-242148 filed Nov. 4, 2011, the entire contents of which is incorporated herein by reference.

### TECHNICAL FIELD

The present invention relates to a process for producing an N-acyl amino acid represented by the formula (1) (hereinafter, sometimes referred to as an N-acyl amino acid (1)):

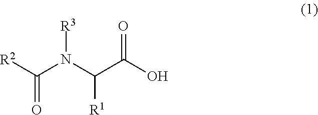

wherein, each of R¹, R² and R³ groups independently represents a hydrogen atom, an optionally-substituted hydrocarbon group or an optionally-substituted heterocyclic group;
by reacting an aldehyde compound represented by the formula (2) (hereinafter, sometimes referred to as an aldehyde compound (2)):

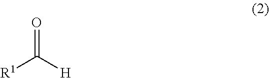

wherein, R¹ is the same as defined above, an amide compound represented by the formula (3) (hereinafter, sometimes referred to as an amide compound (3)):

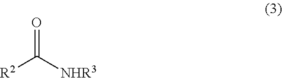

wherein, R² and R³ are the same as defined above, and carbon monoxide. The N-acyl amino acid (1) is useful as, for example, a raw material of pharmaceuticals, agricultural chemicals, or methionine.

### BACKGROUND ART

As a process for producing the N-acyl amino acid (1) by reacting the aldehyde compound (2), the amide compound (3) and carbon monoxide, for example, there have been known a process comprising that the aldehyde compound (2), the amide compound (3), a solvent and a cobalt compound have been placed in a reactor in advance, and are reacted under pressurized carbon monoxide and hydrogen (JP 2008-501737A and JP 2008-501738A), a process comprising that the amide compound (3), the solvent and the cobalt compound are placed in a reactor in advance, and then the aldehyde compound (2) and the solvent are supplied to the reactor under pressurized carbon monoxide and hydrogen to react them (JP 2008-501737A and JP 2008-501738A), and the like.

### SUMMARY OF THE INVENTION

However, a satisfaction cannot be necessarily obtained from the above conventional processes in terms of a yield of the N-acyl amino acid (1).

Then, an object of the present invention is to provide a process for producing the N-acyl amino acid (1) in a good yield.

The present inventors intensively studied and, as the result, the present invention which can attain above object has been accomplished. That is, the present invention provides a process for producing an N-acyl amino acid represented by the formula (1):

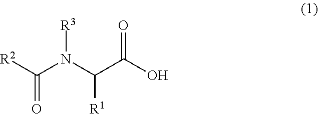

wherein, each of R¹, R² and R³ independently represents a hydrogen atom, an optionally-substituted hydrocarbon group or an optionally-substituted heterocyclic group;
by reacting an aldehyde compound represented by the formula (2):

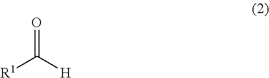

wherein, R¹ is the same as defined above, an amide compound represented by the formula (3):

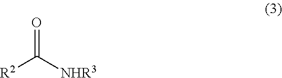

wherein, R² and R³ are the same as defined above, and carbon monoxide,
characterized in that the aldehyde compound represented by the formula (2), the amide compound represented by the formula (3) and a solvent are supplied to a reactor in which a solvent, a cobalt compound, hydrogen and carbon monoxide have been placed in advance.

According to the present invention, the N-acyl amino acid (1) can be produced in a good yield.

### DESCRIPTION OF EMBODIMENTS

In the present invention, an aldehyde compound represented by the formula (2):

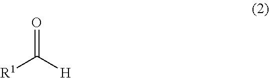

wherein, R¹ represents a hydrogen atom, an optionally-substituted hydrocarbon group or an optionally-substituted heterocyclic group [aldehyde compound (2)],
an amide compound represented by the formula (3):

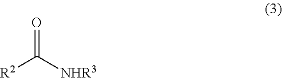

wherein, each of R² and R³ independently represents a hydrogen atom, an optionally-substituted hydrocarbon group or an optionally-substituted heterocyclic group [amide compound (3)], and carbon monoxide are reacted in a solvent in a reactor in the presence of a cobalt compound and hydrogen.

Examples of a hydrocarbon group of the optionally-substituted hydrocarbon group in the formula (2) and the formula (3) include an alkyl group, an alkenyl group, an alkynyl group, a cycloalkyl group, a cycloalkenyl group, an aryl group, and the like. As the alkyl group, an alkyl group having a carbon number of 1-24 is preferable, and examples thereof include a methyl group, an ethyl group, a propyl group, an isopropyl group, a butyl group, an isobutyl group, a s-butyl group, a t-butyl group, a pentyl group, a hexyl group, a heptyl group, an octyl group, a nonyl group, a decyl group, a undecyl group, a dodecyl group, a tridecyl group, a tetradecyl group, a pentadecyl group, a hexadecyl group, a heptadecyl group, an octadecyl group, a nonadecyl group, an icosyl group, an eicosyl group, a henicosyl group, a heneicosyl group, a docosyl group, a tricosyl group, a tetracosyl group, and the like. As the alkenyl group, an alkenyl group having a carbon number of 2-24 is preferable, and examples thereof include a vinyl group, an allyl group, a 2-methylallyl group, an isopropenyl group, a 1-propenyl group, a 1-butenyl group, a 2-butenyl group, a 3-butenyl group, a 1-methyl-1-propenyl group, a 1-methyl-2-propenyl group, a 2-methyl-1-propenyl group, a 2-methyl-2-propenyl group, a 1-pentenyl group, a 2-pentenyl group, a 3-pentenyl group, a 4-pentenyl group, a 1-methyl-1-butenyl group, a 2-methyl-1-butenyl group, a 3-methyl-1-butenyl group, a 1-methyl-2-butenyl group, a 2-methyl-2-butenyl group, a 3-methyl-2-butenyl group, a 1-hexenyl group, a 2-hexenyl group, a 3-hexenyl group, a 4-hexenyl group, a 5-hexenyl group, a 1-methyl-1-pentenyl group, a 2-methyl-1-pentenyl group, a 4-methyl-3-pentenyl group, a 2-ethyl-1-butenyl group, a 2-heptenyl group, a 2-octenyl group, a 2-nonenyl group, a 2-decenyl group, a 2-undecenyl group, a 2-dodecenyl group, a 2-tridecenyl group, a 2-tetradecenyl group, a 2-pentadecenyl group, a 2-hexadecenyl group, a 2-heptadecenyl group, a 2-octadecenyl group, a 2-nonadecenyl group, a 2-icosenyl group, a 2-eicosenyl group, a 2-henicosenyl group, 2-heneicosenyl group, a 2-docosenyl group, a 2-tricosenyl group, a 2-tetracosenyl group, and the like. As the alkynyl group, an alkynyl group having a carbon number of 2-24 is preferable, and examples thereof include an ethynyl group, a 1-propynyl group, a 2-propynyl group, a 1-butynyl group, a 2-butynyl group, a 3-butynyl group, a 1-methyl-2-propynyl group, a 1-pentynyl group, a 2-pentynyl group, a 3-pentynyl group, a 4-pentynyl group, a 1-methyl-3-butynyl group, a 2-methyl-3-butynyl group, a 1-hexynyl group, a 2-hexynyl group, a 3-hexynyl group, a 4-hexynyl group, a 5-hexynyl group, a 2-heptynyl group, a 2-octynyl group, a 2-nonynyl group, a 2-decynyl group, a 2-undecynyl group, a 2-dodecynyl group, a 2-tridecynyl group, a 2-tetradecynyl group, a 2-pentadecynyl group, a 2-hexadecynyl group, a 2-heptadecynyl group, a 2-octadecynyl group, a 2-nonadecynyl group, a 2-icosynyl group, a 2-eicosynyl group, a 2-henicosynyl group, a 2-heneicosynyl group, a 2-docosynyl group, a 2-tricosynyl group, a 2-tetracosynyl group, and the like. As the cycloalkyl group, a cycloalkyl group having a carbon number of 3-8 is preferable, and examples thereof include a cyclopropyl group, a cyclobutyl group, a cyclopentyl group, a cyclohexyl group, a cycloheptyl group, a cyclooctyl group, and the like. As the cycloalkenyl group, a cycloalkenyl group having a carbon number of 3-8 is preferable, and examples thereof include a cyclopropenyl group, a cyclobutenyl group, a cyclopentenyl group, a cyclohexenyl group, a cycloheptenyl group, a cyclooctenyl group, and the like. Examples of an aryl group include a phenyl group, a naphthyl group, an anthranyl group, a phenanthryl group, a tolyl group, a xylyl group, and the like.

In the formula (2) and the formula (3), examples of a heterocyclic group in the optionally-substituted heterocyclic group include a heteroaryl group, a heteroaralkyl group, and the like. As the heteroaryl group, a heteroaryl group having a carbon number of 3-9 is preferable, and examples thereof include a pyridyl group, a quinonyl group, a pyrrolyl group, an imidazolyl group, a furyl group, an indolyl group, a thienyl group, an oxazolyl group, and the like. As the heteroaralkyl group, a heteroaralkyl group having a carbon number of 5-10 is preferable, and examples thereof include a pyridylmethyl group, a quinonylmethyl group, an indolylmethyl group, a furylmethyl group, a pyrrolylmethyl group, and the like.

The hydrocarbon group and the heterocyclic group as mentioned above may be substituted. When the hydrocarbon group is an alkyl group, an alkenyl group or an alkynyl group, examples of its substituent include a halogen atom such as fluorine, chlorine and bromine; a cycloalkyl group having a carbon number of 3-6 such as a cyclopropyl group, a 1-methylcyclopropyl group, a cyclobutyl group, a cyclopentyl group, a 1-methylcyclopentyl group and a cyclohexyl group; an alkoxy group having a carbon number of 1-4 such as a methoxy group, an ethoxy group, a propoxy group, an isopropoxy group, a butoxy group, a s-butoxy group, an isobutoxy group and a t-butoxy group; a thioalkoxy group having a carbon atom of 1-4 such as a thiomethoxy group, a thioethoxy group, a thiopropoxy group and a thiobutoxy group; an alkenyloxy group having a carbon number of 3-4 such as an allyloxy group, a 2-propenyloxy group, a 2-butenyloxy group and a 2-methyl-3-propenyloxy group; an aralkyloxy group having a carbon number of 7-20; an aryl group having a carbon number of 6-18 such as a phenyl group, a naphthyl group, an anthracenyl group and a phenanthryl group; an aryloxy group such as a phenyloxy group and a naphthyloxy group; an alkanoyl group having a carbon number of 2-7; an aryloyl group having a carbon number of 7-19; an alkanoylamino group having a carbon number of 2-7; an alkylsulfonylamino group having a carbon number of 1-6; an alkoxycarobonylamino group having a carbon number of 2-6; a benzylcarbonylamino group; an arylsulfonylamino group having a carbon number of 6-18; an aminocarbonyl group; an alkoxycarbonyl group having a carbon number of 1-6, and the like. When the hydrocarbon group is the alkyl group, examples of the alkyl group substituted with an aryl group having a carbon number of 6-18 include an aralkyl group such as a benzyl group, a phenethyl group, a 3-phenylpropyl group, a benzhydryl group, a trityl group, a triphenylethyl group, a (1-naphthyl)methyl group, a (2-naphthyl)methyl group, and the like.

When the hydrocarbon group as mentioned above is a cycloalkyl group, a cycloalkenyl group or an aryl group, examples of its substituent include the halogen atom as mentioned above, a cycloalkyl group having a carbon number of 3-6, an alkoxy group having a carbon number of 1-4, a thioalkoxy group having a carbon number of 1-4, an alkenyloxy group having a carbon number of 3-4, an aralkyloxy group having a carbon number of 7-20, an aryl group having a carbon number of 6-18, an aryloxy group, an alkanoyl group having a carbon number of 2-7, an aryloyl group having a carbon number of 7-19, an alkanoylamino group having a carbon number of 2-7, an alkylsulfonylamino group having a carbon number of 1-6, an alkoxycarbonylamino group having a carbon number of 2-6, a benzylcarbonylamino group, an arylsulfonylamino group having a carbon number of 6-18, an aminocarbonyl group, an alkoxycarbonyl group having a carbon number of 1-6, an alkyl group having a carbon number of 1-6 such as a methyl group, an ethyl group, a propyl group, an isopropyl group, a butyl group, an isobutyl group, a s-butyl group, a t-butyl group, a pentyl group and a hexyl group, an alkenyl group having a carbon number of 2-6 such as a vinyl group, a 1-propenyl group, a 2-propenyl group, a 1-butenyl group, a 2-butenyl group, a 3-butenyl group, a 1-methyl-2-propenyl group, a 2-methyl-2-propenyl group, a 1-pentenyl group, a 2-pentenyl group, a 3-pentenyl group, a 4-pentenyl group, a 1-methyl-2-butenyl group, a 2-methyl-2-butenyl group, a 1-hexenyl group, a 2-hexenyl group, a 3-hexenyl group, a 4-hexenyl group and a 5-hexenyl group, an aralkyl group having a carbon number of 7-20 such as a benzyl group, a phenethyl group and a naphthylmethyl group, and the like. Examples of the substituent in the heterocyclic group include the halogen atom as mentioned above, a cycloalkyl group having a carbon number of 3-6, an alkoxy group having a carbon number of 1-4, a thioalkoxy group having a carbon number of 1-4, an alkenyloxy group having a carbon number of 3-4, an aralkyloxy group having a carbon number of 7-20, an aryl group having a carbon number of 6-18, an aryloxy group, an alkanoyl group having a carbon number of 2-7, an aryloyl group having a carbon number of 7-19, an alkanoylamino group having a carbon number of 2-7, an alkylsulfonylamino group having a carbon number of 1-6, an alkoxycarbonylamino group having a carbon number of 2-6, a benzylcarbonylamino group, an arylsulfonylamino group having a carbon number of 6-18, an aminocarbonyl group, an alkoxycarbonyl group having a carbon number of 1-6, the alkyl group having a carbon number of 1-6 as mentioned above, an alkenyl group having a carbon number of 2-6, an aralkyl group having a carbon number of 7-20, and the like.

Examples of the aldehyde compound (2) include formaldehyde, acetaldehyde, propionaldehyde, butyraldehyde, valeraldehyde, 3-(methylthio)propionaldehyde, 2-ethylhexanal, isobutyraldehyde, furfural, crotonaldehyde, acrolein, benzaldehyde, substituted benzaldehyde, phenylacetaldehyde, 2,4-dihydroxyphenylacetaldehyde, glyoxalic acid, α-acetoxypropionaldehyde, and the like. Particularly, the process of the present invention is advantageously utilized when 3-(methylthio)propionaldehyde is used as a raw material.

Examples of the amide compound (3) include acetamide, benzamide, propionamide, N-methyl acetamide, fatty acid amide, acrylamide, cinnamamide, phenylacetamide, acetanilide, urea, and the like. Particularly, the process of the present invention is advantageously utilized when acetamide is used as a raw material.

An amount of the amide compound (3) to be used is usually 1.00 mole or more and preferably 1.05-2.00 moles relative to 1 mole of the aldehyde compound (2).

Examples of the solvent used in the reaction include an organic solvent, an ionic liquid, and the like. Examples of the organic solvent include an alcoholic solvent such as methanol, ethanol and isopropyl alcohol; an ether solvent such as 1,4-dioxane, tetrahydrofuran, ethylene glycol dimethyl ether, t-butyl methyl ether, dibutyl ether and cyclopentyl methyl ether; N-methylpyrrolidinone, N-ethylpyrrolidinone, 1,3-dimethyl-2-imidazolidinone, dimethylsulfoxide, acetone, ethyl acetate, butyl acetate, acetonitrile, benzonitrile, sulfolane, N,N-dimethylformamide, N,N-dimethylacetamide, toluene, acetic acid, and the like. Particularly, 1,4-dioxane is preferable. Furthermore, only one kind of the solvent may be used, or two or more kinds of the solvent may be used together.

An amount of the solvent to be used is preferably 0.50-20.0 folds (by weight) and more preferably 2.0-10.0 folds (by weight) relative to that of the aldehyde compound (2). When two or more kinds of the solvent are used together, a total amount thereof may be within a range as mentioned above.

In the present invention, a cobalt compound is used as a catalyst. Examples of the cobalt compound include a divalent cobalt compound such as cobalt chloride (II), cobalt bromide (II), cobalt iodide (II), cobalt nitrate (II), cobalt sulfate (II) and cobalt acetate (II); a cobalt carbonyl complex such as octacarbonyldicobalt (0) and tetracobalt dodecacarbonyl (0); a cobalt phosphine complex such as dibromo bis(triphenylphosphine)cobalt (II) and tetrakis(trimethylphosphine)methylcobalt (I), and the like. Particularly, the cobalt carbonyl complex is preferable in terms of a yield of the N-acyl amino acid (1) to be obtained. Furthermore, the cobalt compound may be used after being shaped, being adhered on a carrier, or being immobilized on a polymer compound.

An amount of the cobalt compound to be used is usually 0.00010-0.80 mole and preferably 0.010-0.090 mole relative to 1 mole of the aldehyde compound (2).

In the present invention, the reaction as mentioned above is conducted in the presence of hydrogen. A ratio of hydrogen and carbon monoxide to be used is preferably 1/1-9/1, more preferably 1/1-4/1, and yet more preferably 2/1-3/1 in a molar ratio of carbon monoxide to hydrogen (carbon monoxide/hydrogen).

Moreover, in the present invention, the reaction as mentioned above may be conducted in the presence of water. An amount of water to be used is preferably 0.1-2.0 moles and more preferably 0.5-1.5 mole relative to 1 mole of the aldehyde compound (2).

In the present invention, the reaction as mentioned above may be conducted in the presence of an acid. Examples of the acid include an inorganic acid such as sulfuric acid, nitric acid, hydrogen chloride and phosphoric acid; an organic acid such as toluenesulfonic acid, methanesulfonic acid and trichloroacetic acid; an ion-exchange resin, and the like. Particularly, sulfuric acid is preferable. When the inorganic acid or the organic acid is used, an amount thereof to be used is preferably 0.001-0.02 mole relative to 1 mole of the aldehyde compound (2).

Next, a reaction mode of the reaction as mentioned above will be explained. In the present invention, first, the solvent, the cobalt compound, hydrogen and carbon monoxide are introduced. An order of introducing those components into the reactor is not particularly limited, but introducing the solvent and the cobalt compound followed by hydrogen and carbon monoxide is preferable. After introducing those components into the reactor, the aldehyde compound (2), the amide compound (3) and the solvent are supplied to the reactor. The aldehyde compound (2), the amide compound (3) and the solvent may be supplied alone (so-called, co-feed) or as a mixture thereof but, preferably, the aldehyde compound (2), the amide compound (3) and the solvent are supplied as a mixed solution. A total amount of the aldehyde compound (2) may be supplied to the reactor together with the amide compound (3) and the solvent, or a part thereof may be placed in the reactor in advance, and then the rest may be supplied to the reactor together with the amide compound (3) and the solvent. In addition, similarly, a total amount of the amide compound (3) maybe supplied to the reactor together with the aldehyde compound (2) and the solvent, or a part thereof may be placed in the reactor in advance, and then the rest may be supplied to the reactor together with the aldehyde compound (2) and the solvent. In the present invention, a total amount of the aldehyde compound (2), a total amount of the amide compound (3) and the solvent are preferably supplied to the reactor, in which the solvent, the cobalt compound, hydrogen and carbon monoxide has been placed. In addition, an amount of the solvent to be placed in the reactor in advance is preferably 30-90% by weight relative to a total amount of the solvent. That is, the aldehyde compound (2), the amide compound (3) and a rest of the solvent (i.e., 10-70% by weight of a total amount of the solvent) are preferably supplied to the reactor in which 30-90% by weight of the solvent relative to a total amount thereof, the cobalt compound, hydrogen and carbon monoxide are placed in advance. In addition, when the reaction as mentioned above is conducted in the presence of water, water may be introduced to the reactor in advance, or may be supplied together with the aldehyde compound (2), the amide compound (3) and the solvent, or a part of water may be placed in the reactor in advance and then a rest thereof may be supplied to the reactor together with the aldehyde compound (2), the amide compound (3) and the solvent. Water is preferably supplied together with the aldehyde compound (2), the amide compound (3) and the solvent. When the reaction as mentioned above is conducted in the presence of an acid, the acid may be introduced to the reactor in advance, or it may be supplied to the reactor together with the aldehyde compound (2), the amide compound (3) and the solvent, or a part of the acid is placed in the reactor in advance and then a rest thereof may be supplied to the reactor together with the aldehyde compound (2), the amide compound (3) and the solvent. The acid is preferably introduced into the reactor in advance.

Furthermore, each of the aldehyde compound (2) and the amide compound (3) maybe supplied to the reactor continuously without interval or intermittently with a predetermined interval. In addition, a start of supplying the aldehyde compound (2) and the amide compound (3), and completion of supplying the aldehyde compound (2) and the amide compound (3) may not be necessarily accorded, and may be shifted in a range so long as the effect of the present invention is not deteriorated.

The aldehyde compound (2) is desirably supplied in a cooled state. This allows not only suppression of a reaction between two aldehyde compound (2) molecules (aldol condensation), but also suppression of a byproduct derived from this condensation product. The temperature of the aldehyde compound (2) to be cooled depends upon a kind thereof, but is usually about −20-5° C.

The reaction temperature is usually 60-140° C. and preferably 80-120° C. In addition, reaction pressure may be ordinary pressure, but the reaction may be conducted under a pressurized condition of preferably 0.1-25 MPa and more preferably 8-18 MPa in absolute pressure. When the reaction as mentioned above is conducted under pressure, pressure may be applied with a mixed gas of hydrogen and carbon monoxide, and an inert gas such as a nitrogen gas and a helium gas may used for adjustment of reaction pressure. The reaction as mentioned above may be conducted either of a continuous, semi-continuous, or batch process.

Thus, the N-acyl amino acid [N-acyl amino acid (1)] represented by the formula (1):

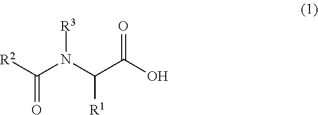

wherein, R¹, R² and R³ are the same as defined above, can be produced in a good yield. A post-reaction procedure of a reaction mixture containing the N-acyl amino acid (1) obtained after the reaction may be properly selected, and the product maybe used for various kinds of use, if necessary, after washing, or purification such as distillation and crystallization.

### EXAMPLES

An example embodiment of the present invention is illustrated below, but does not limit the present invention.

In the Examples, an amount of acetylmethionine [the compound represented by the formula (1) wherein R¹ is a 2-thiomethoxyethyl group, R² is a methyl group, and R³ is a hydrogen atom] was analyzed with a liquid chromatography to calculate a yield.

### Example 1

To a stainless reactor equipped with a thermocouple, a stirrer, a gas supply line and a liquid supply line, 5.40 g (0.016 mol) of octacarbonyl dicobalt(0)[Co₂(CO)₈] and 39.86 g of 1,4-dioxane (38.7% by weight relative to a total amount of 1, 4-dioxane) were placed and stirred, and a mixed gas of carbon monoxide and hydrogen [carbon monoxide/hydrogen=2.3/1 (molar ratio)] was introduced to a gas phase portion in the reactor such that pressure in the reactor became 13 MPa (gauge pressure). Then, a temperature in the reactor was elevated to 68-72° C. while stirring was continued. At this time, pressure in the reactor was 13 MPa (gauge pressure). Then, to the reactor, a mixed solution of 31.57 g (0.30 mole) of 3-(methylthio)propionaldehyde [a compound represented by the formula (2) wherein R¹ is a 2-thiomethoxyethyl group], 18.08 g (0.30 mole) of acetamide [a compound represented by the formula (3) wherein R² is a methyl group and R³ is a hydrogen atom], 63.14 g of 1,4-dioxane (61.3% by weight relative to a total amount of 1,4-dioxane) and 5.40 g of water was added dropwise over 1.5 hour. After addition, the temperature in the reactor was held at 68-72° C. for 4 hours while stirring was continued, and then it was cooled to 5-35° C. to obtain 158.3 g of an acetyl methionine solution in 1,4-dioxane. A liquid chromatography analysis of the solution revealed that a yield of acetyl methionine relative to 3-(methylthio)propionaldehyde was 85.5%.

### Comparative Example 1

To a stainless reactor equipped with a thermocouple, a stirrer, a gas supply line and a liquid supply line, 17.36 g (0.17 mole) of 3-(methylthio)propionaldehyde, 9.95 g (0.17 mole) of acetamide, 2.97 g (0.0087 mole) of octacarbonyl dicobalt (0) and 56.65 g of 1,4-dioxane (100% by weight relative to a total amount of 1,4-dioxane) were placed and stirred, and a mixed gas of carbon monoxide and hydrogen [carbon monoxide/hydrogen=2.3/1 (molar ratio)] was introduced to a gas phase portion in the reactor such that pressure in the reactor became 13 MPa (gauge pressure). Then, a temperature in the reactor was elevated to 68-72° C. while stirring was continued. At this time, pressure in the reactor was 13 MPa (gauge pressure). Then, the temperature in the reactor was held at 68-72° C. for 4 hours while stirring was continued, and then it was cooled to 5-35° C. to obtain 84.2 g of an acetyl methionine solution in 1,4-dioxane. A liquid chromatography analysis of the solution revealed that a yield of acetyl methionine relative to 3-(methylthio)propionaldehyde is 73.0%.

### Comparative Example 2

To a stainless reactor equipped with a thermocouple, a stirrer, a gas supply line and a liquid supply line, 9.95 g (0.17 mole) of acetamide, 2.97 g (0.0087 mole) of octacarbonyl dicobalt (0) and 56.65 g of 1, 4-dioxane (100% by weight relative to a total amount of 1,4-dioxane) were placed and stirred, and a mixed gas of carbon monoxide and hydrogen [carbon monoxide/hydrogen=2.3/1 (molar ratio)] was introduced to a gas phase portion in the reactor such that pressure in the reactor became 13 MPa (gauge pressure). Then, a temperature in the reactor was elevated to 68-72° C. while stirring was continued. At this time, pressure in the reactor was 13 MPa (gauge pressure). Then, 17.36 g (0.17 mole) of 3-(methylthio)propionaldehyde was added dropwise to the reactor over 1.0 hour. After addition, the temperature was held at 68-72° C. for 4 hours while stirring was continued, and then it was cooled to 5-35° C. to obtain 87.8 g of an acetyl methionine solution in 1,4-dioxane. A liquid chromatography analysis of the solution revealed that a yield of acetyl methionine relative to 3-(methylthio)propionaldehyde was 81.5%.

1. A process for producing an N-acyl amino acid represented by the formula 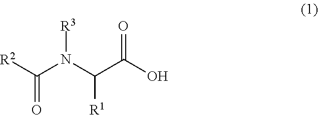
wherein, each of R¹, R² and R³ independently represents a hydrogen atom, an optionally-substituted hydrocarbon group or an optionally-substituted heterocyclic group;
by reacting an aldehyde compound represented by the formula (2): 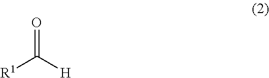
wherein, R¹ is the same as defined above,
an amide compound represented by the formula (3): 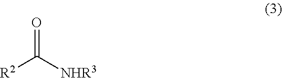
wherein, R² and R³ are the same as defined above, and
carbon monoxide, in a solvent in a reactor in the presence of a cobalt compound and hydrogen,
characterized in that the aldehyde compound represented by the formula (2), the amide compound represented by the formula (3) and a solvent are supplied to the reactor in which the solvent, the cobalt compound, hydrogen and carbon monoxide have been placed in advance.

2. The process according to claim 1, wherein the aldehyde compound represented by the formula (2), the amide compound represented by the formula (3) and 10-70% by weight of the solvent relative to a total amount of the solvent are supplied to the reactor in which a rest of the solvent, the cobalt compound, hydrogen and carbon monoxide have been placed in advance.

3. The process according to claim 1, wherein the reaction is conducted further in the presence of water, and further water is supplied.

4. The process according to claim 1, wherein the aldehyde compound represented by the formula (2) is 3-(methylthio)propionaldehyde.

5. The process according to claim 1, wherein the amide compound represented by the formula (3) is acetamide.

6. The process according to claim 1, wherein the solvent is 1,4-dioxane.

7. The process according to claim 1, wherein the cobalt compound is a cobalt carbonyl complex.
